(19) United States
(12) Patent Application Publication
(10) Pub. No.: US 2014/0276015 A1
(43) Pub. Date: Sep. 18, 2014

(54) DEVICES AND METHODS FOR IMAGING AND CROSSING OCCLUDED VESSELS

(71) Applicant: VOLCANO CORPORATION, San Diego, CA (US)

(72) Inventor: Chester Whiseant, Marysville, CA (US)

(73) Assignee: VOLCANO CORPORATION, San Diego, CA (US)

(21) Appl. No.: 14/205,903

(22) Filed: Mar. 12, 2014

Related U.S. Application Data
(60) Provisional application No. 61/781,368, filed on Mar. 14, 2013.

(51) Int. Cl. A61B 8/00; A61B 5/00; A61M 25/09; A61B 8/12

(52) U.S. Cl. CPC A61B 8/445; A61B 8/12; A61B 5/0095; A61B 5/6852; A61M 25/09
USPC 600/425; 600/407; 600/467

(57) ABSTRACT

The invention provides devices and methods for crossing total chronic occlusions. In certain aspects, a device for imaging a vessel includes an elongate body defining a first lumen and comprising a distal end; a housing operably associated with the distal end and comprising a forward-looking imaging element; and a member at least partially disposed within the first lumen of the elongate body; the member configured to extend beyond the distal end of the elongate body to advance into an occluded vessel.

### RELATED APPLICATION

The present application claims the benefit of and priority to U.S. Provisional No. 61/781,368, filed Mar. 14, 2013, which is incorporated by reference in its entirety.

### TECHNICAL FIELD

This application generally relates to devices and methods for imaging and crossing occluded vessels.

### BACKGROUND

Cardiovascular disease frequently arises from the accumulation of atheromatous deposits on inner walls of vascular lumen, particularly the arterial lumen of the coronary and other vasculature, resulting in a condition known as atherosclerosis. These deposits can have widely varying properties, with some deposits being relatively soft and others being fibrous and/or calcified. In the latter case, the deposits are frequently referred to as plaque. These deposits can restrict blood flow, which in severe cases can lead to myocardial infarction.

In certain instances, the level of occlusion in a vessel is significant enough to completely block blood flow to portions of the vasculature distal to the blockage. This type of blockage is known as a chronic total occlusion. Revascularization of vessels with chronic total occlusions poses significant challenges due the inability of angiography to image/visualize the occluded vessel. Intraluminal imaging techniques have been employed to provide some insight into the occluded vessel. Specifically, forward-looking imaging catheters are often used to guide revascularization of the occluded vessel. These devices are particularly advantageous because they allow a physician see what is in front of the catheter, and also allow imaging in areas which cannot be crossed with the catheter (e.g. a total chronic occlusion).

A limiting factor of forward looking intraluminal catheters is one of girth. Typically, forward-looking imaging elements required must span across a diameter of the imaging device so that an active face (for sending and receiving imaging signals) is at least partially facing the forward direction. While forward looking catheter provides some insight into the occluded vessel, the forward-looking catheter is often unable to cross the occlusion itself.

During a typical procedure, the forward looking catheter is advanced to the site of the target occlusion over a guidewire under fluoroscopy. The forward looking catheter takes an image to assess the position of the guidewire with respect to a proximal cap of the occlusion. If the positioning is good, the clinician attempts to penetrate the proximal cap with the guidewire. Ideally, the wire and catheter are then advanced into the lesion, using the forward-viewing imaging capability to verify that the wire and catheter are remaining within the true lumen of the vessel. However, the diameter of the forward looking imaging device relative to the vessel combined with the density or calcification of the lesion often render it impossible to advance the wire unsupported into the lesion, or to advance the forward looking imaging catheter. When this occurs, a course of action often pursued is to withdraw the imaging device in exchange for a non-imaging micro-catheter. This requirement to withdraw the device and exchange adds procedure time, cost, risks loss of wire position and completely removes the ability to image within the vessel whatsoever.

### SUMMARY

Devices and methods of the invention provide for a forward-looking catheter with a telescoping micro-catheter that is able to advance a guidewire supported into an occluded lesion and assist in penetration of the lesion. Particular advantages of the invention include visualization of the crossing event, more support for the guidewire to prevent incidental passage into a false lumen, and elimination of the exchange of multiple devices.

In certain aspects of the invention, a device for imaging a vessel includes an elongate body defining a first lumen and comprising a distal end. A housing is operably associated with the distal end and includes a forward looking imaging element. A member is at least partially disposed within the first lumen of the elongate body, and is able to translate within and out of the lumen. For example, the member is configured to extend beyond the distal end of the elongate body to advance into an occluded vessel.

The housing of the imaging catheter of the invention may be formed as part of the elongate body or may be separate from and coupled to the elongate body. In certain embodiments, the housing forms an atraumatic tip of the elongate body. The housing may include a lumen that is axially aligned with the lumen of the elongate body. In certain embodiments, a forward looking imaging element is located on the housing and configured to image an object in an imaging plane in front of the elongate body. For example, a forward-looking imaging element is located on a distal end of an intraluminal device and is able to image an object within a forward imaging plane, which is a distance in front of the imaging element. In certain embodiments, an active face of the forward looking imaging element is angled. The elongate body may include one or more coil layers defining the lumen of the elongate body. Preferably, the elongate body includes two coil layers. In certain embodiment, one or more signal lines to the forward looking imaging element of the inner member are disposed within the lumen and surrounded by the one or more coil layers. The elongate body may be configured to rotate to provide a forward looking cone of visualization in front of the elongate body with the imaging element.

As discussed, devices of the invention include an elongate body and a member moveably disposed within the elongate body. In certain aspects, the member of the device is configured to support a guidewire into and through an occluded vessel. This prevents the guidewire from failing to cross into the true lumen of the vessel or perforation of the vessel wall due to travel through a false lumen. The member of the device may include a coil shaft. The coil shaft may include one or more coil layers. In one embodiment, the coil shaft is a single coil layer. The coil shaft of the member may be operably coupled to a drive shaft configured to provide rotational and translational motion of the member. In certain embodiments, the member may include an imaging element. For example, the imaging element may be a ring-transducer wrapped around the circumference of the member or a single or linear array transducer located along a side of the member. The signal lines for the imaging element may be embedded in the coil shaft and drive shaft. The member may include a lumen that is co-axial with the lumen of the elongate body in which a guidewire may extend there through. A length of the member may only be a portion of a length of the elongate body. For example, a length of the member comprising the coil may only be a portion of a length of the elongate body.

In certain embodiments, the member further includes a cap. The cap may also define a lumen for receiving a guidewire there through. The cap may be formed as part of the member or may be formed separate from the member and coupled to a distal end of the member. In one embodiment, a dimension of at least one portion of the cap is greater than a dimension of the lumen of the elongate body. In this manner, the cap prevents the member from retracting into the elongate body during use. In addition, the cap may comprise one or more flutes or wedges. The one or more flutes may be configured to facilitate penetration of an occluded lesion in a vessel. In one embodiment, the flutes are shaped (e.g. in a spiral fashion) such that rotation of the member during forward penetration encourages movement of the member within the occlusion.

### BRIEF DESCRIPTION OF THE DRAWINGS

FIG. 1 is a beamformer of forward-looking imaging planes.

FIGS. 2-4 are isometric views showing different imaging planes generated by a forward-looking and side-viewing catheters.

FIG. 5 depicts a prior art forward looking catheter and its imaging plane.

FIG. 6 depicts a guidewire moving within a false lumen of an occlusion.

FIGS. 7A-7E depict a device of the invention according to certain embodiments.

FIG. 8 depicts an alternative embodiment of a device of the invention.

FIG. 9 is a simplified diagrammatic view of the device coupled to drive actuation systems and an interface module.

FIG. 10 is a simplified illustration of a rotary drive actuation system.

FIGS. 11A-11D depict a method of using a device according to certain embodiment to cross an occluded vessel.

FIG. 12 is a system diagram according to certain embodiments.

### DETAILED DESCRIPTION

The invention generally relates to a forward-looking intraluminal catheter having an elongate body with telescoping inner member disposed therein. The telescoping inner member is configured to extend beyond a distal end of the elongate body in order to provide support to a guidewire attempting to penetrate an occluded vessel segment (such as a chronic total occlusion). In addition, the telescoping inner member may be used to facilitate penetration of the occluded vessel segment and provide continued support for the guidewire while crossing the occluded vessel segment. Furthermore, the telescoping inner member could be used to enlarge the path through the occluded segment in order to allow the elongate body member to pass through the occluded segment.

According to certain aspects, a forward-looking intraluminal catheter of the invention is used to image an intraluminal surface. In certain embodiments, the intraluminal surface being imaged is a surface of a body lumen. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, uterus and fallopian tubes, structures of the urinary tract including urinary collecting ducts, renal tubules, ureter, and bladder, and structures of the head and neck and pulmonary system including sinuses, parotid, trachea, bronchi, and lungs.

Particularly, the forward-looking intraluminal catheter is useful for imaging an object disposed in front of the catheter, such as plaque accumulation. The forward-looking intraluminal catheter may be used to image a chronic total occlusion in front the catheter, and provide guidance for an operator to visualize a guidewire crossing the occlusion. The forward looking catheter of the invention allows for identification and differentiation of a true vessel lumen from a fall vessel lumen, and facilitates guidewire entry into the true lumen of an occluded vessel.

According to certain embodiments, a catheter includes a forward-looking intraluminal imaging element located on a distal end of the catheter body. Typically, the imaging element is a component of an imaging assembly. Any imaging assembly may be used with devices and methods of the invention, such as optical-acoustic imaging apparatus, intravascular ultrasound (IVUS) or optical coherence tomography (OCT). The imaging element is used to send and receive signals to and from the imaging surface that form the imaging data. In one embodiment, the forward-looking imaging element is disposed on and/or within a housing on the distal end of the elongate body. The housing may formed as part of and integral with the distal end of the elongate body (i.e. the housing forms the distal end) or the housing may be separate from and coupled to the distal end of the elongate body.

Some of the ultrasonic imaging catheters currently in use are “side viewing” devices which produce B-mode images in a plane which is perpendicular to the longitudinal axis of the catheter and passes through the transducer. That plane can be referred to as the B-mode lateral plane and is illustrated in FIG. 4. Devices of the invention may include a “side viewing” B mode imaging element.

Forward looking imaging elements image an object a distance in front of the imaging element. For example, there are devices that produce a C-mode image plane as illustrated in FIG. 2. The C-mode image plane is perpendicular to the axis of an intraluminal device and spaced in front of the imaging element. The imaging signals are transmitted at an arbitrary angle from an axis of the imaging element to image a cross-section in front of the imaging element. Other forward viewing devices produce a B-mode image in a plane that extends in a forward direction from the imaging element and parallel to the axis of the catheter. FIG. 3 exemplifies a B-Mode forward imaging plane. FIG. 1 shows the beamformer geometry and imaging planes of forward C-mode and forward B-mode imaging elements.

The imaging element shown in FIGS. 2 and 3 (as well as FIG. 8) include a ring-array of transducers that circumscribes the housing (or distal end) of the intraluminal device. The ring array includes a set of transducers that image the tissue with ultrasound energy (e.g., 20-50 MHz range) and a set of transducers that collect the returned energy (echo) to create an intravascular image. The array is arranged in a cylindrical pattern, allowing the imaging assembly to image 360° inside a vessel. In some embodiment, the transducers producing the energy and the transducers receiving the echoes are the same elements, e.g., piezoelectric elements. A ring-array of transducers is able to obtain a cross-sectional image of the vessel without rotating the elongate body having the imaging element disposed thereon.

Alternatively, a forward looking imaging assembly may include an imaging element located on a portion of the distal end, such as imaging element 23 shown in FIG. 5. The imaging element 1 may be angled, such as forward slanting angle towards the distal tip, in order to manipulate the forward imaging plane. Particularly, the forward imaging element may have a 35° to 45° forward slant. The imaging element may include one or more transducers that image tissue with ultrasound energy and a set of transducers that collected the returned energy (echo) to create an intravascular image. For cross-sectional imaging with imaging element 23, the elongate body rotates 180 degrees to produce a tomographic image. The imaging assembly is rotated and manipulated longitudinally by a drive cable coupled to an elongate body.

Examples of forward-looking ultrasound assemblies are described in U.S. Pat. Nos. 7,736,317, 6,780,157, and 6,457,365, and in Yao Wang, Douglas N. Stephens, and Matthew O'Donnellie, “Optimizing the Beam Pattern of a Forward-Viewing Ring-Annular Ultrasound Array for Intravascular Imaging”, Transactions on Ultrasonics, Rerroelectrics, and Frequency Control, vol. 49, no. 12, December 2002. Examples of forward-looking optical coherence tomography assemblies are described in U.S. Publication No. 2010/0220334, Fleming C. P., Wang H., Quan, K. J., and Rollins A. M., “Real-time monitoring of cardiac radio-frequency ablation lesion formation using an optical coherence tomography forward-imaging catheter.” J. Biomed. Opt. 15, (3), 030516-030513 ((2010)), and Wang H, Kang W, Carrigan T, et al; In vivo intracardiac optical coherence tomography imaging through percutaneous access: toward image-guided radio-frequency ablation. J. Biomed. Opt. 0001; 16(11):110505-110505-3. doi:10.1117/1.3656966. Examples of photoacoustic assemblies that may be forward or side viewing are described in U.S. Pat. Nos. 6,659,957 and 7,527,594, 7,245.789, 7447,388, 7,660,492, 8,059,923 and in U.S. Patent Publication Nos. 2008/0119739, 2010/0087732 and 2012/0108943.

In certain aspects, an imaging assembly includes both side-viewing and forward-looking capabilities. Side-viewing imaging elements image a cross-section of the vessel directly parallel to imaging element. These imaging elements are known as “side viewing” devices that produce B-mode images in a plane that is perpendicular to the longitudinal axis of the intraluminal device and passes through the imaging element. The imaging plane of B-mode side-viewing images is shown in FIG. 4. For side-viewing cross-sectional imaging, the shortened distal tips of the invention are advantageous because the shortened tip significantly reduces the distance between the cross-sectional imaging plane and distal tip of the catheter, without sacrificing protection of the imaging element. As a result, an operator can obtain images with the side-viewing imaging element right next to a blockage, in difficult tortuous angles, and in bi-furcations. Examples of side-viewing intravascular ultrasound assemblies are describe in, for example, U.S. Pat. Nos. 4,794,931, 5,000,185, 5,243,988, 5,353,798, and 5,375.602. Examples of side-viewing optical coherence tomography assemblies are described in, for example, U.S. Pat. Nos. 7,929,148, 7,577,471, and 6,546,272. Combined side-viewing and forward viewing catheters utilize different frequencies that permit the imaging assembly to isolate between forward looking imaging signals and side viewing imaging signals. For example, the imaging assembly is designed so that a side imaging port is mainly sensitive to side-viewing frequencies and a forward viewing imaging port is mainly sensitive to forward viewing frequencies. Example of this type of imaging element is described in U.S. Pat. Nos. 7,736,317, 6,780,157, and 6,457,365.

FIG. 5 illustrates a prior art forward viewing catheter in more detail. As shown in FIG. 5, prior art catheter 1 includes an elongate body 21 disposed within a catheter sheath 21. A distal end of the elongate body includes an imaging element 23. A guidewire 15 is extending through a lumen of the elongate body. For imaging, the elongate body rotates within the catheter sheath 21 to obtain images within the imaging area 27.

Although the prior art catheter 1 may be used to image a occlusion 20 located in front of the imaging element 23, the catheter 1 is unable to facilitate guidewire entry into the true lumen 25. FIG. 6 depicts a common problem with prior art catheters. The catheter 1 is able to image the occlusion 20 in front of the catheter 1, but is too large to enter the proximal cap of the total chronic occlusion 20. As a result, a guidewire 15 must travel unsupported into the occlusion. This often results in the guidewire moving sub-intimal and forming a false lumen (as shown in FIG. 6). The false lumen risks perforation of the vessel and decreases the ability of other devices to avoid the created false lumen in order to pass into the true lumen 25. In addition, an unsupported guidewire 15 traveling through the occlusion increases risk of vessel wall perforation.

Catheters overcome the limitations of current forward looking imaging catheters discussed in the background section and with regard to FIG. 6 by providing a telescoping inner member. The telescoping inner member extends distally from a distal end of the elongate body of a catheter of the invention to provide support to a guidewire entering and crossing an occluded vessel. With the additional support from the inner telescoping member, catheters of the invention are able to prevent creation of a false lumen by a guidewire. In addition, the inner telescoping member assists in forming a path to the true lumen within the occlusion. The path created within the occlusion by the inner telescoping member increases the chances that the elongate body of the catheter will likewise be able to cross the occlusion despite its girth. This allows for imaging within and beyond the occlusion.

FIG. 7A-7E illustrate a forward-looking catheter 100 of the invention. Referring to FIG. 7A, a catheter 100 of the invention includes elongate body 101 with a housing 107 and an imaging element 105 disposed on and/or within the housing 107. The imaging element 105 may be any of the imaging elements or imaging assemblies described above, e.g. forward looking imaging element combined forward/side viewing imaging elements. In preferred embodiments and as shown, the imaging element 105 is an ultrasound transducer configured to provide forward cross-sectional images upon rotation. The imaging element 105 is angled with a forward slant (e.g. 35° to 50°). The imaging element 107 is coupled to one or more signal lines 124. The signal line 124 is disposed within the lumen 130 of the elongate body 101.

The elongate body 101 may have a coating 112 covering a coil shaft 120 (FIG. 7A). The coil shaft 120 is flexible, but retains enough rigidity to transmit rotation from a proximal end of the coil shaft 120 to the distal end 135 of elongate body 101. The coil shaft 120 may be coupled to a stiffer proximal shaft (not shown). The proximal shaft couples to a drive shaft to provide rotation to the elongate body 101. Alternatively, the coil shaft 120 may couple directly to a drive shaft, which provides rotation to the elongate body 101. Optionally, the elongate body 101 is disposed within a catheter sheath 102. The coil shaft 120 may include one or more layers. In one embodiment, the coil shaft 102 is a duel layer coil. An exemplary coil shaft 120 is a torque coil provided by Asahi Intecc. However, any coil capable of transmitting rotation from a proximal end of the coil shaft 120 to the distal end 135 of the coil shaft 120 is suitable for use. The coil shaft 102 defines the lumen 130 of the elongate body 101.

The housing 107 may be formed as part of and integral with the distal end 130 of the elongate body 101 (i.e. the housing forms the distal end) or the housing may be separate from and coupled to the distal end 130 of the elongate body 101. The housing 107 includes the imaging element 105 and an atraumatic tip 111. The atramatic tip 111 prevents inadvertent damage to a vessel wall. In certain embodiments, the atraumatic tip 111 is tapered. This allows the elongate body to move more easily through the bends of the vasculature. In addition, the housing 107 defines a lumen 132 (FIG. 7E). The lumen 132 of the housing 107 is co-axially aligned with a lumen 130 of the elongate body 101. In certain embodiments, the lumen 132 of the housing 107 is the same as the lumen 130 of the elongate body.

The forward-looking catheter 100 further includes an inner member 108 disposed within the lumen 130 of elongate body 101 and the housing 132. The inner member 108 defines a lumen co-axially aligned with the lumens elongate body 101 and the housing 132. The guidewire 15 is able to pass through the lumens of the elongate body 101, housing 132, and the inner member 108. The inner member 108 is configured to rotate and translate with respect to the elongate body 109. The inner member 108 includes a coil shaft 122. The coil shaft 122 of the inner member 108 is flexible, but retains enough rigidity to transmit rotation from a proximal end of the coil shaft 122 to the distal end 150 of inner member 108. The coil shaft 122 may couple to a more rigid proximal shaft, which couples to a drive shaft. Alternative, the coil shaft 122 may couple directly to the drive shaft. The drive shaft may be used to impart rotational and translational motion to the inner member 108. The coil shaft 122 of the inner member 108 may include one or more layers. In one embodiment, the coil shaft 122 of the inner member 108 is a single layer coil. An exemplary coil shaft 122 is an Asahi Intecc Actone single layer torque coil.

Referring now to FIG. 7C, the inner member 108, according to certain embodiments, may include an imaging element 110. The imaging element 110 may be coupled to one or more signal lines (not shown) embedded in the coil shaft 122 or disposed within the coil shaft 122 (which is surrounding the signal lines). The imaging element 110 may include any imaging element described above, including forward-looking or side viewing imaging elements. In particular aspects, the imaging element 110 is a ring-array transducer (such as the imaging element shown in FIG. 8). With the imaging element 110, the inner member 108 may obtain images of the occlusion as the inner element facilities crossing of the occlusion.

In certain embodiments, the inner member 108 further includes a cap 109. In certain embodiments, a cross-sectional dimension of the cap 109 is larger the cross-sectional dimensions of the lumen 132 of the housing and the lumen 130 of the elongate body 101 and/or lumen 132 of the housing 107. This prevents the inner member 108 from retracting within the elongate body during use. In certain embodiments, the cap includes a first portion 170 having a first dimension and a second portion 160 having a second dimension small than the first dimension (as shown in FIGS. 7D and 7E). The second portion 160 of the cap 109 couples to the coil shaft 122 of the inner member 108. The cap 109 may further include one or more flutes 115. The flutes 115 are shown in more detail in FIG. 7B. In certain embodiments, the flutes 115 are spiral cuts formed into the cap 109 (such as the spiral cuts in a screw). As shown in FIG. 7E, the flutes 115 are formed into the first portion 170 of the cap 109. The flutes 115 are configured to assist in driving and stabilizing the inner member, while rotated, into an occlusion (similar to a screw driving in to a wall). The flutes 115 may also be used to open a larger channel within the occlusion as the inner member 108 advanced and retracted into the occlusion. FIG. 7D shows the inner member 108 in an extended position and FIG. 7E shows the inner member 108 in a retracted position.

FIG. 8 depicts an alternative embodiment of a catheter of the invention. Instead of the rotating forward imaging element shown in FIG. 7, the catheter 400 comprises a ring-array imaging element 408. The ring-array imaging element 408 includes a plurality of transducer elements that are arranged in a cylindrical array. The ring-array imaging element 408 is configured to provide forward images without the need for rotation of the elongate body 402. The ring array imaging element 408 is provided at the distal end 410 of the catheter 400, with a connector 424 located at the proximal end of the catheter. Like the catheter shown in FIGS. 7A-7E, the catheter shown in FIG. 8 includes an inner member 404 configured to rotate and translate with respect to the elongate body 402. As shown, a guidewire 15 is extending through the elongate body 402. An example of a ring array forward imaging element is described in U.S. Pat. No. 7,736,617.

FIG. 9 depicts a schematic overview of the mechanisms for imparting rotation to the elongate body 101 and the inner member 108 according to certain embodiments. As shown, the elongate body 101 couples to a first rotary/linear drive actuation system 233, and inner member 108 couples to a second rotary/linear drive actuation system 222. Any actuation systems known in the art that are configured to impart rotational and/or linear movement of a catheter shaft are suitable for use in methods and systems of the invention. The first actuation system 233 and second actuation system 222 may be one device. Alternatively, the first actuation system 233 is separate from the second actuation system 222. For embodiments with an imaging element disposed on a rotating shaft, such as the elongate body, the actuation system should include an optical/electrical rotary joint in order to allow rotation of the signal line with the imaging element while maintaining a stationary electrical/optical connection at a proximal end (e.g. for the connection at an interface module 244). Rotary joints connect two signal lines, one signal line that is stationary and proximal to the rotary joint and another signal line that is rotatable and distal to the rotary joint. The actuation systems 222, 233 can be configured so that the inner member 108 rotates along with and in sync with the elongate body 101. Alternatively, the actuations systems 222, 233 can be configured to provide rotational motion to the elongate body 101 that is separate from the rotational motion of the inner member 108.

FIG. 10 outlines an exemplary drive actuation system for imparting rotation motion to the coil shaft of the elongate body or the coil shaft of the inner member. The actuation system includes a rotary motor, a gear, a rotary joint, a drive shaft, and a signal line. The rotary joint maintains a signal line stationary at the proximal end, while the coil shaft and distal portion of the signal are rotated by the gear and motor. The signal line may couple to an interface system or module 425.

The interface module 425 (FIG. 9) provides a plurality of user interface controls that can be manipulated by a user. The interface module 425 also communicates via the signal line with one or more imaging element of the elongate body 101 and/or the inner member 108 by sending and receiving signals to and from the imaging elements. The interface module 425 can receive, analyze, and/or display information received from the imaging elements.

FIGS. 11A-11D exemplify a method of crossing an occluded vessel using devices of the invention. As shown in FIG. 11A, the elongate body 101 of the device 100 is approaching a chronic total occlusion 20 in vessel 10. The elongate body 101 is riding over guidewire 15. For crossing the occlusion 20, the elongate body 101 is rotated to provide an image of the occlusion for an operator. The obtained image may be used to determine an appropriate entry point into the occlusion 20, e.g. a point directed towards the path for the true lumen 25 of the vessel. Once the entry point is determined, the inner member 108 may be extended to provide support to the guidewire 15 as the guidewire 15 attempts to break into the occlusion 20 (as shown in FIG. 11B). For heavily calcified, fibrous occlusions, the wire may attempt to deviate due to the pressure exerted in a failed attempt to enter the occlusion 20, which may lead to vessel 10 penetration. With the added support from inner member 108, the wire 15 is unable to deviate due to a failed attempt, and the pressure exerted on the occlusion 20 is more focused. The elongate member 101 may be used to image the guidewire 15 and the inner member 108 at various stages of the advancement and/or during the advancement for real-time visualization.

After the guidewire 15 enters the occlusion 20, the inner member 108 may also be extended into the occlusion 20 (as shown in FIG. 11C). The cap 109 of the inner member 108 may be used to enlarge the pathway to the true lumen 25 as the inner member 108 is advanced into the occlusion 20. In certain embodiments and as shown, the inner member 108 is rotated to facilitate its advancement into the occlusions. One or more flutes (previously-discussed) on the cap assist with advancement of the inner member 108 and expansion of the pathway in the occlusion 20. In addition, the advancement of the inner member 108 provides continued support for the guidewire 15 as moves within the true lumen 25. Once the guidewire 15 and/or the inner member 108 successfully crossed the occlusion 20, the elongate body 101 may be advanced into the occlusion 20.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

In some embodiments, a user interacts with a visual interface to view images from the imaging system. Input from a user (e.g., parameters or a selection) are received by a processor in an electronic device. The selection can be rendered into a visible display. An exemplary system including an electronic device is illustrated in FIG. 12. As shown in FIG. 12, an imaging engine 859 of the imaging assembly communicates with host workstation 433 as well as optionally server 413 over network 409. The data acquisition element 855 (DAQ) of the imaging engine receives imaging data from one or more imaging element. In some embodiments, an operator uses computer 449 or terminal 467 to control system 400 or to receive images. An image may be displayed using an I/O 454, 437, or 471, which may include a monitor. Any I/O may include a keyboard, mouse or touchscreen to communicate with any of processor 421, 459, 441, or 475, for example, to cause data to be stored in any tangible, nontransitory memory 463, 445, 479, or 429. Server 413 generally includes an interface module 425 (also shown in FIG. 9) to effectuate communication over network 409 or write data to data file 417.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server 413), a middleware component (e.g., an application server), or a front-end component (e.g., a client computer 449 having a graphical user interface 454 or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a portion of file 417 that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A device for imaging a vessel, the device comprising
an elongate body defining a first lumen and comprising a distal end;
a housing operably associated with the distal end and comprising a forward-looking imaging element; and
a member at least partially disposed within the first lumen of the elongate body; the member configured to extend beyond the distal end of the elongate body to advance into an occluded vessel.

2. The device of claim 1, wherein the elongate body comprises a dual coil layer.

3. The device of claim 2, wherein elongate body comprises an outer sheath surrounding the dual coil layer.

4. The device of claim 2, wherein the dual coil layer defines the first lumen and surrounds the member.

5. The device of claim 1, wherein the member comprises a second imaging element.

6. The device of claim 1, wherein the member further comprises a cap coupled to a distal portion of the member.

7. The device of claim 1, wherein a diameter of the cap is greater than a diameter of the first lumen.

8. The device of claim 6, wherein the cap includes at least one flute configured to facilitate advancement of the member into the occluded vessel.

9. The device of claim 1, wherein the member defines a second lumen co-axially aligned with the first lumen.

10. The device of claim 5, wherein the second imaging element comprises a ring-transducer array surrounding a distal portion of the member.

11. The device of claim 5, wherein the forward looking imaging element or the second imaging element is capable of collecting data selected from the group consisting of optical coherence tomography data, ultrasound data, and photoacoustic data.

12. The device of claim 1, wherein a length of the member is a portion of a length of the elongate member.

13. The device of claim 1, wherein the member is rotatable.

14. The device of claim 1, wherein the housing forms an atraumatic tip of the elongate body.
